(19) United States
(12) Patent Application Publication
(10) Pub. No.: US 2022/0291010 A1
(43) Pub. Date: Sep. 15, 2022

(54) APPARATUS AND METHOD FOR MEASURING SALINITY, AND SYSTEM FOR MANAGING SALINITY

(71) Applicant: Yamada Giken Co., Ltd., Fukui (JP)

(72) Inventors: Naoto MIKAMI, Tokyo (JP); Hideo SASAKI, Tokyo (JP); Yukihiro NAKANO, Tokyo (JP); Yutaka SUYA, Fukui (JP); Takeo YAMADA, Fukui (JP); Takayuki NAKAYAMA, Fukui (JP); Toru TOKUNAGA, Fukui (JP)

(21) Appl. No.: 17/678,561

(22) Filed: Feb. 23, 2022

(30) Foreign Application Priority Data
Mar. 11, 2021 (JP) 2021-038808

(51) Int. Cl. G01C 21/36; B60R 16/08; G01N 33/18

(52) U.S. Cl. CPC G01C 21/3691; B60R 16/08; G01N 33/18

(57) ABSTRACT

A concentration sensor 36 is disposed behind a wheel 22, and for sensing salinity concentration of water bounced up by the wheel 22. A front nozzle 34 is disposed ahead the wheel 22, and for sprinkling water toward an area of the road surface on which the wheel 22 is to pass. A rear nozzle 35 is disposed between the wheel 22 and the front nozzle 34, and for sprinkling water toward the wheel.

### TECHNICAL FIELD

The present invention is related to an apparatus and method for measuring salinity, especially, on a road surface on which a vehicle travels, and a system for managing the salinity.

### BACKGROUND ART

EP 1602913 A1 discloses a method including: sprinkling water toward a dry road surface to dissolve salinity on the road surface; bouncing up the water containing the dissolved salinity by a wheel of a vehicle; and measuring salinity concentration of the bounced water to measure salinity on the road surface.

### SUMMARY OF INVENTION

### Technical Problem

In the method disclosed in EP 1602913 A1, a relatively large amount of water is sprinkled in order to bounce up a large amount of water enough to sense salinity concentration. This reduces accuracy of the salinity measurement. Also, this may cause the sprinkled water to freeze.

To prevent this, EP 1602913 A1 discloses sprinkling salt water in a case that the atmospheric temperature is low. However, accuracy of the salinity measurement is further reduced in a case that the concentration of the sprinkled salt water is not adequately managed.

### Solution to Problem

An apparatus is for measuring salinity on a road surface. The apparatus is mounted in a vehicle having a wheel. The vehicle travels on the road surface. The vehicle is an automobile, for example. The apparatus includes the following components.
(1) A concentration sensor. The concentration sensor is disposed behind the wheel to be watered by water bounced up by the wheel. The wheel is a tire, for example. The wheel may be a drive wheel, or may be a non-drive wheel. The concentration sensor senses salinity concentration of the water bounced up by the wheel. The concentration sensor may be one disclosed in EP 1602913 A1, or may have other configurations.
(2) A front nozzle. The front nozzle is disposed ahead the wheel. The front nozzle sprinkles water toward the road surface, especially toward an area on which the wheel is to pass, to dissolve salinity on the road surface in the sprinkled water. The front nozzle sprinkles a minimum amount of water required for dissolving the salinity on the road surface to prevent reduction of accuracy of salinity measurement. Also, this enables to prevent the sprinkled water from freezing. Thus, there is no need to sprinkle salt water. This improves accuracy of salinity measurement.
(3) A rear nozzle. The rear nozzle is disposed between the wheel and the front nozzle. The rear nozzle sprinkles water toward the wheel to wet the wheel. The water attached to the wheel is mixed with the water on the road surface. Thereby, an amount of the water bounced up by the wheel becomes large enough to sense salinity concentration by the concentration sensor.

The apparatus may include an electric control unit. The electric control unit may be a computer, for example. The electric control unit may compute an amount of the salinity on the road surface. The computation may be performed based on the salinity concentration sensed by the concentration sensor and an amount of the water sprinkled by the front nozzle and the rear nozzle.

A system is for managing salinity on a road surface. The system includes the following components.
(1) The above-described apparatus.
(2) A display. The display displays a dangerous area on a map. The dangerous area is determined that an amount of salinity in the dangerous area is less than a predetermined threshold. The determination is performed based on the amount of the salinity measured by the apparatus and a position of the vehicle at a time when the concentration sensor senses the salinity concentration. The predetermined threshold is determined by considering an amount of salinity required to prevent the road surface from freezing to assure safety.

A method is for measuring salinity on a road surface. A vehicle has a wheel, and travels on the road surface. The method includes the following steps.
(1) Water is sprinkled from a front nozzle. The front nozzle is disposed ahead the wheel. The water is sprinkled toward the road surface, especially, an area on which the wheel is to pass, to dissolve salinity on the road surface in the sprinkled water. A minimum amount of water required for dissolving the salinity on the road surface is sprinkled to prevent reduction of accuracy of salinity measurement. Also, this enables to prevent the sprinkled water from freezing. Thus, there is no need to sprinkle salt water. This improves accuracy of salinity measurement.
(2) Water is sprinkled from a rear nozzle. The rear nozzle is disposed between the wheel and the front nozzle. The water is sprinkled toward the wheel to wet the wheel. The water attached to the wheel is mixed with the water on the road surface. Thereby, an amount of the water bounced up by the wheel becomes large enough to sense salinity concentration by the concentration sensor.
(3) Salinity concentration of water bounced up by the wheel is sensed by a concentration sensor. The concentration sensor is disposed behind the wheel to be watered by water bounced up by the wheel. The wheel is a tire, for example. The wheel may be a drive wheel, or may be a non-drive wheel. The concentration sensor may be one disclosed in EP 1602913 A1, or may have other configurations.

The method may include a step for computing an amount of the salinity. The computation may be performed based on the salinity concentration measured by the concentration sensor and an amount of the water sprinkled by the front nozzle and the rear nozzle.

The method may include the following steps.
(1) A position of the vehicle is sensed at a time when the concentration sensor measures the salinity concentration.
(2) A dangerous area in which the amount of the salinity is less than a predetermined threshold is determined. The determination may be performed based on the computed amount of the salinity and the measured position. The predetermined threshold may be determined by considering an amount of salinity required to prevent the road surface from freezing to assure safety.

The method may include a step for displaying the determined dangerous area on a map.

### Advantageous Effects of the Invention

The wheel is wet by the water sprinkled from the rear nozzle. Thereby, even though an amount of the water sprinkled from the front nozzle toward the road surface is small, an amount of the water bounced up by the wheel is large enough to sense salinity concentration by the concentration sensor. Since the amount of the water sprinkled toward the road surface can be small, an amount of the salinity on the road surface can be precisely measured. Also, since the amount of the water sprinkled toward the road surface can be small, there is no fear that the sprinkled water freezes. Thus, there is no need to sprinkle salt water, and thereby, an amount of the salinity on the road surface can be precisely measured.

### BRIEF DESCRIPTION OF DRAWINGS

FIG. 1 is a block diagram showing an example of a system according to the present invention; and

FIG. 2 is a flowchart showing an example of a method according to the present invention.

### DESCRIPTION OF EMBODIMENT

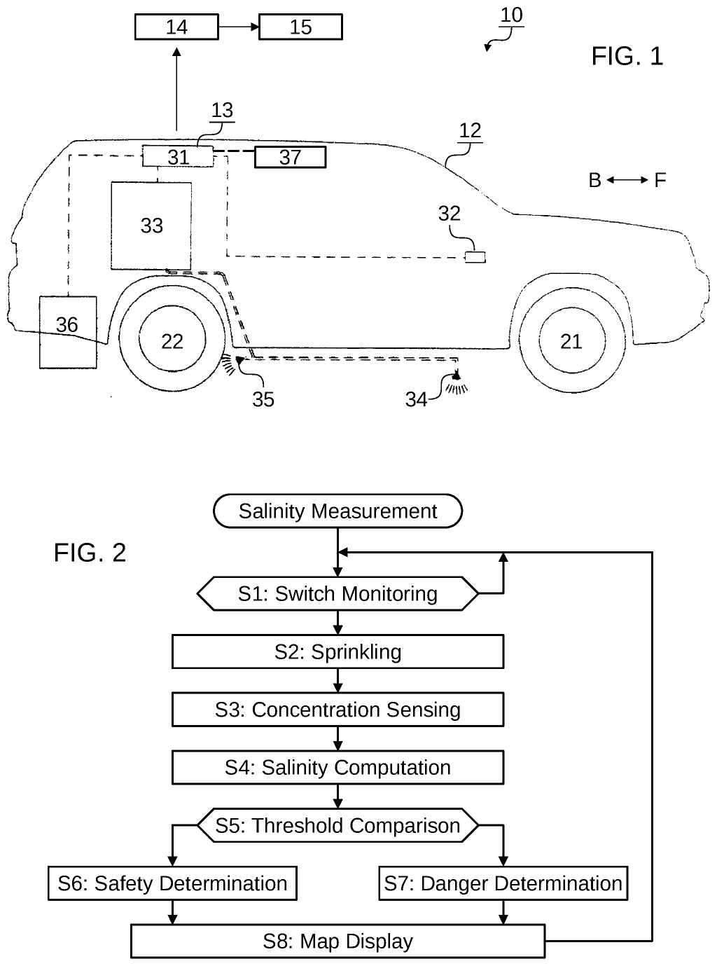

Referring FIG. 1, a salinity management system 10 measures salinity on a dry road surface. The salinity management system 10 includes a vehicle 12, a salinity measurement apparatus 13, a server computer 14, and a liquid crystal display unit 15, for example.

The vehicle 12 travels on the road surface. The vehicle 12 normally moves toward F direction, but it can also move toward B direction. The vehicle 12 is self-propelled, but it may be trailed by a self-propelled vehicle.

The vehicle 12 has two front tires 21 and two rear tires 22, for example. The tires 21, 22 roll on the road surface.

The salinity measurement apparatus 13 is mounted in the vehicle 12. The salinity measurement apparatus 13 measures salinity on the road surface. The salinity measurement apparatus 13 includes an electric control unit (ECU) 31, a switch 32, a water tank 33, a front nozzle 34, a rear nozzle 35, a concentration sensor 36, and a positioning unit 37, for example.

The ECU 31 is a computer or other electric circuits, for example. The ECU 31 is installed in the vehicle 12. The ECU 31 controls sprinkling and measurement.

The switch 32 is installed at an assistant driver's seat in the vehicle 12, for example. The switch 32 is a toggle switch, for example. When the switch 32 is turned on, the sprinkling is started. When the switch 32 is turned off, the sprinkling is stopped. The switch 32 may be an automatic return switch which is automatically turned off when a predetermined time passes after the switch is turned on. Or, the switch 32 may be a push switch which is on only while the switch is pushed.

For example, the ECU 31 monitors a state of the switch 32. When detecting that the switch 32 is on, the ECU 31 issues a sprinkling command. When detecting that the switch 32 is off, the ECU 31 stops issuing the sprinkling command. In a case that the switch 32 is a push switch, the ECU 31 may continue to issue the sprinkling command until a predetermined time passes after the switch 32 is turned on.

The water tank 33 is installed in the vehicle 12, especially in a luggage space, for example. The water tank 33 stores water to be sprinkled. The water stored in the water tank 33 is fresh water containing no salt.

The front nozzle 34 is installed under a chassis in the vehicle 12, for example. The front nozzle 34 communicates with the water tank 33 to sprinkle the water stored in the water tank 33. The front nozzle 34 is directed downward to sprinkle the water toward the road surface. The front nozzle 34 is directed to sprinkle the water toward a direction perpendicular to the road surface, for example.

The front nozzle 34 is disposed at a position in F direction from one of the rear tires 22. Hereinafter, the one of the rear tires 22 is called “measurement wheel.” When the vehicle 12 travels on the road surface toward F direction, the measurement wheel 22 passes on an area onto which the front nozzle 34 sprinkles the water.

When the switch 32 is on, the front nozzle 34 sprinkles the water. When the switch 32 is off, the front nozzle 34 stops sprinkling the water. For example, the ECU 31 issues the sprinkling command, a valve disposed at a middle of a pipe communicating between the water tank 33 and the front nozzle 34 is opened to supply water to the front nozzle 34. When the ECU 31 stops issuing the sprinkling command, the valve is closed.

The rear nozzle 35 is installed under the chassis in the vehicle 12, for example. The rear nozzle 35 communicates with the water tank 33 to sprinkle the water stored in the water tank 33. The rear nozzle 35 is disposed at a position between the measurement wheel 22 and the front nozzle 34. The rear nozzle 35 is directed to the measurement wheel 22 to sprinkle the water toward the measurement wheel 22. The rear nozzle 35 is directed to sprinkle the water toward a direction downwardly diagonal to a surface of the measurement wheel 22, for example.

A surface of the measurement wheel 22, especially, an area at a front side, is wetted by the water sprinkled from the rear nozzle 35. When the vehicle 12 travels on the road surface toward F direction, the measurement wheel 22 rolls, and thereby, the area wet by the water sprinkled from the rear nozzle 35 touches the road surface.

When the switch 32 is on, the rear nozzle 35 sprinkles the water. When the switch 32 is off, the rear nozzle 35 stops sprinkling the water. For example, the ECU 31 issues the sprinkling command, a valve disposed at a middle of a pipe communicating between the water tank 33 and the rear nozzle 35 is opened to supply water to the rear nozzle 35. When the ECU 31 stops issuing the sprinkling command, the valve is closed.

The concentration sensor 36 is installed under the chassis in the vehicle 12, for example. The concentration sensor 36 senses salinity concentration of water bounced up by the measurement wheel 22. The concentration sensor 36 is disposed at a position in B direction from the measurement wheel 22 to be watered by water bounced up by the measurement wheel 22.

Since the measurement wheel 22 is wet by the water sprinkled from the rear nozzle 35 in advance, a large amount of the water enough to sense salinity concentration by the concentration sensor 36 is bounced up even when an amount of the water sprinkled from the front nozzle 34 is small.

This enables to reduce an amount of the water sprinkled from the front nozzle 34 toward the road surface. This enables to improve accuracy of the salinity measurement. Also, this enables to eliminate fear that the water sprinkled toward the road surface freezes. So, there is no need to sprinkle salt water. Since fresh water is sprinkled toward the road surface, there is no need to correct the measured salinity concentration by subtracting salinity contained in the sprinkled water. This enables to improve accuracy of the salinity measurement.

The concentration sensor 36 senses the salinity concentration once a second, for example. The concentration sensor 36 sends data representing the sensed salinity concentration to the ECU 31.

The ECU 31 receives the data sent from the concentration sensor 36. The ECU 31 computes an amount of salinity on the road surface based on the salinity concentration sensed by the concentration sensor 36 and an amount of the water sprinkled from the front nozzle 34 and the rear nozzle 35. The amount of the water sprinkled from the front nozzle 34 and the rear nozzle 35 may be fixed.

For example, salinity concentration C₁ of the water on the road surface can be represented by the following formula:

C₁=R·S/V,

where R denotes an amount of residual salinity per unit area on the road surface, V denotes a volume of the water per unit time sprinkled from the front nozzle 34, and S denotes an area per unit time on which the water is sprinkled from the front nozzle 34.

And, the salinity concentration C₂ of the water bounced up by the measurement wheel 22 can be represented by the following formula:

C₂=p·C₁,

where p denotes a proportion of the water on the road surface within the water bounced up by the measurement wheel 22. It should be noted that the water bounced up by the measurement wheel 22 contains the water sprinkled from the rear nozzle 35.

Accordingly, the ECU 31 can compute the amount of residual salinity per unit area on the road surface by using the following formula:

R=k·C₂,

where k denotes a predetermined constant, k=V/(p·S).

The positioning unit 37 is installed in the vehicle 12. The positioning unit 37 measures a position of the vehicle 12. The positioning unit 37 may be a receiver of the Global Positioning System (GPS).

The positioning unit 37 measures the position of the vehicle 12 once a second, for example. The positioning unit 37 sends data representing the measured position to the ECU 31.

The ECU 31 receives the data sent from the positioning unit 37. The ECU 31 associates the computed amount of salinity on the road surface with the position of the vehicle 12 at a time when the concentration sensor 36 senses the salinity concentration. The ECU 31 sends data representing the computed amount of salinity on the road surface and data representing the associated position of the vehicle 12 to the server computer 14.

The computation of the amount of the residual salinity on the road surface may be performed by the server computer 14, instead of the ECU 31. In this case, the ECU 31 sends data representing the salinity concentration sensed by the concentration sensor 36, instead of data representing the computed amount of the residual salinity on the road surface, to the server computer 14.

The data may be sent to the server computer 14 in real-time, or may be recorded in the ECU 31 and sent all at once later.

The server computer 14 is disposed apart from the vehicle 12. The server computer 14 communicates with the ECU 31 via a wireless communication, public telecommunication network, or others.

The server computer 14 receives the data sent from the ECU 31. The server computer 14 determines a dangerous area in the road surface, for example. In a case that an amount of residual salinity per unit area is less than a predetermined threshold, there is a risk of the road surface freezing. So, the area is determined as the dangerous area.

For example, the server computer 14 compares the amount of residual salinity with the predetermined threshold. When the amount of residual salinity is smaller than the predetermined threshold, the server computer 14 determines that the associated position of the vehicle 12 is within the dangerous area. When the amount of residual salinity is greater than the predetermined threshold, the server computer 14 determines that the associated position of the vehicle 12 is not within the dangerous area.

The comparison of the amount of residual salinity with the predetermined threshold may be performed by the ECU 31, instead of the server computer 14. In this case, the ECU 31 sends data representing result of the comparison, instead of, or in addition to, the data representing the computed amount of residual salinity, to the server computer 14. Also, the determination of the dangerous area may be performed by the ECU 31, instead of the server computer 14.

The liquid crystal display unit 15 displays a road map. The liquid crystal display unit 15 displays the dangerous area determined by the server computer 14 superposed on the displayed map. For example, the dangerous area is represented in red. This enables to easily grasp the dangerous area, where salt or other anti-freezing agents should be scattered. The liquid crystal display unit 15 may be disposed in a vehicle for scattering anti-freezing agents. The vehicle may be the same as the vehicle 12, or be different from the vehicle 12.

Anti-freezing agents may be automatically scattered. For example, the server computer 14 sends data representing the dangerous area to the vehicle for scattering anti-freezing agents. When the vehicle passes the dangerous area, the vehicle automatically scatters anti-freezing agents.

Referring to FIG. 2, a salinity measurement method includes a switch monitoring step S1, a sprinkling step S2, a concentration sensing step S3, a salinity computation step S4, a threshold comparison step S5, a safety determination step S6, a danger determination step S7, and a map display step S8.

In the salinity measurement method, the process starts from the switch monitoring step S1.

In the switch monitoring step S1, the ECU 31 monitors the state of the switch 32. In a case that the switch 32 is off, the process repeats the switch monitoring step S1. In a case that the switch 32 is on, the process proceeds to the sprinkling step S2.

In the sprinkling step S2, the front nozzle 34 and the rear nozzle 35 sprinkle the water stored in the water tank 33. Then, the process proceeds to the concentration sensing step S3.

In the concentration sensing step S3, the concentration sensor 36 senses salinity concentration of the water bounced up by the measurement wheel 22. Then, the process proceeds to the salinity computation step S4.

In the salinity computation step S4, the ECU 31 computes an amount of residual salinity per unit area on the road surface. Then, the process proceeds to the threshold comparison step S5.

In the threshold comparison step S5, the server computer 14 compares the amount of residual salinity with a predetermined threshold. In a case that the amount of residual salinity is larger than the threshold, the process proceeds the safety determination step S6. In a case that the amount of residual salinity is smaller than the threshold, the process proceeds the danger determination step S7.

In the safety determination step S6, the server computer 14 determines that the position of the vehicle 12 at a time when the concentration sensor 36 senses the salinity concentration is within a safe area. Then, the process proceeds to the map display step S8.

In the danger determination step S7, the server computer 14 determines that the position of the vehicle 12 at a time when the concentration sensor 36 senses the salinity concentration is within a dangerous area. Then, the process proceeds to the map display step S8.

In the map display step S8, the liquid crystal display unit 15 displays the dangerous area on a map. Then, the process returns to the switch monitoring step S1.

The above-described embodiments are examples to make it easier to understand the present invention. The present invention is not limited to the examples, and includes any modified, altered, added, or removed variations, without departing from the scope of the claims attached herewith. This can be easily understood by persons skilled in the art.

### REFERENCE SIGNS LIST

10: Salinity management system; 12: Vehicle; 13: salinity measurement apparatus; 14: server computer; 15: liquid crystal display unit; 21: front tire; 22: rear tire; 31: ECU; 32: switch; 33: water tank; 34: front nozzle; 35: rear nozzle; 36: concentration sensor; 37: positioning unit; S1: switch determination; S2: sprinkling; S3: concentration sensing; S4: salinity computation; S5: threshold comparison; S6: safety determination; S7: danger determination; and S8: map display.

1. An apparatus for measuring salinity on a road surface, wherein the apparatus is mounted in a vehicle having a wheel, the vehicle travels on the road surface, and the apparatus comprises:
a concentration sensor disposed behind the wheel, and for sensing salinity concentration of water bounced up by the wheel;
a front nozzle disposed ahead the wheel, and for sprinkling water toward an area of the road surface on which the wheel is to pass; and
a rear nozzle disposed between the wheel and the front nozzle, and for sprinkling water toward the wheel.

2. The apparatus of claim 1, further comprising
an electric control unit for computing an amount of the salinity on the road surface based on the salinity concentration sensed by the concentration sensor and an amount of the water sprinkled by the front nozzle and the rear nozzle.

3. A system for managing salinity on a road surface, comprising:
the apparatus of claim 1; and
a display for displaying a dangerous area on a map, the dangerous area being determined that an amount of the salinity in the dangerous area is less than a predetermined threshold, based on the amount of the salinity measured by the apparatus and a position of the vehicle at a time when the concentration sensor senses the salinity concentration.

4. A method for measuring salinity on a road surface, wherein a vehicle having a wheel travels on the road surface, the method comprises:
sprinkling water from a front nozzle disposed ahead the wheel toward an area of the road surface on which the wheel is to pass;
sprinkling water from a rear nozzle disposed between the wheel and the front nozzle toward the wheel; and
sensing salinity concentration of water bounced up by the wheel by a concentration sensor disposed behind the wheel.

5. The method of claim 4, further comprising
computing an amount of the salinity on the road surface based on the salinity concentration sensed by the concentration sensor and an amount of the water sprinkled by the front nozzle and the rear nozzle.

6. The method of claim 5, further comprising:
measuring a position of the vehicle at a time when the concentration sensor senses the salinity concentration; and
determining a dangerous area in which the amount of the salinity is less than a predetermined threshold, based on the computed amount of the salinity and the measured position.

7. The method of claim 6, further comprising
displaying the determined dangerous area on a map.
